(19) United States
(12) Patent Application Publication
(10) Pub. No.: US 2016/0355632 A1
(43) Pub. Date: Dec. 8, 2016

(54) AQUEOUS DISPERSIONS OF CATIONIC FILM-FORMING POLYURETHANES

(71) Applicant: LAMBERTI SPA, Albizzate (IT)

(72) Inventors: Vito ALANZO, Castano Primo (IT); Gabriele COSTA, Novate Milanese (IT); Dario CONTI, Castano Primo (MI) (IT); Giuseppe LI BASSI, Gavirate (VA) (IT)

(73) Assignee: LAMBERTI SPA, Albizzate (IT)

(21) Appl. No.: 15/242,004

(22) Filed: Aug. 19, 2016

Related U.S. Application Data
(63) Continuation-in-part of application No. 12/989,369, filed on Oct. 22, 2010.

(51) Int. Cl. C08G 18/08; A61Q 5/06; C08G 18/10; C08G 18/78; D06M 15/572; C08G 18/42; C09D 175/06; C09D 175/08; D21H 19/12; D06N 3/14; A61K 8/87; C08G 18/48

(52) U.S. Cl. CPC C08G 18/0809; A61K 8/87; A61Q 5/06; C08G 18/10; C08G 18/785; C08G 18/4854; C08G 18/4238; C08G 18/0866; C09D 175/06; C09D 175/08; D21H 19/12; D06N 3/146; D06M 15/572; A61K 2800/5426

(57) ABSTRACT

Stable aqueous dispersions of film-forming cationic polyurethanes are prepared by extending with water a cationic prepolymer obtained from aliphatic diisocyanates, aliphatic tertiary amines having two hydroxyl groups that react with —NCO groups, linear polyols and methanesulfonic acid.

### CROSS-REFERENCE TO RELATED APPLICATION

This application in a continuation-in-part of U.S. patent application Ser. No. 12/989,369, filed Oct. 22, 2010, which is a 371 National Phase Entry of PCT/EP09/54874, filed Apr. 23, 2009, the disclosure of which is fully incorporated herein by reference.

### BACKGROUND OF THE INVENTION

The present invention relates to aqueous dispersions of film-forming cationic polyurethanes and to their use in the preparation of cosmetics and personal care products and of formulations for treating leather, textiles and paper.

According to one of the fundamental aspect of the present invention the procedure for the preparation of the above aqueous dispersions is described.

Aqueous dispersions of cationic polyurethanes which are capable of producing films (film-forming polyurethanes) are known; their preparation and use in many industrial fields, such as in the field of cosmetics, textiles, leather and paper, is described in several patents.

By way of example, U.S. Pat. No. 3,388,087 describes the preparation of aqueous dispersions of quaternized polyurethanes which are useful for the preparation of flexible films that are useful in the cosmetic industry as ingredients of hair fixative lacquers.

U.S. Pat. No. 3,480,592 describes the preparation of aqueous dispersions of polyurethanes containing salified tertiary amine groups; the films obtained from these polyurethanes have applications that are similar to those of U.S. Pat. No. 3,388,087.

More recently, U.S. Pat. No. 6,335,003 reports the preparation of polyurethanes containing salified tertiary amine groups; the polyurethanes of U.S. Pat. No. 6,335,003 are different from the polyurethanes of U.S. Pat. No. 3,480,592 because of their high amine number.

US 2004/0052753 describes elastic polyurethanes containing salified tertiary amine groups and their use in cosmetic compositions.

At least hypothetically, aqueous dispersions of cationic film-forming polyurethanes can constitute a valid alternative to the commonly used urea-formaldehyde resins, the use of which has become critical due to more and more strict regulatory issues on formaldehyde residues in products for personal use (textiles included).

Nonetheless, relatively few products of these kinds are available on the market.

The main problem which is encountered in the preparation of aqueous dispersions of film-forming cationic polyurethanes is the balance of their components that shall guarantee, at the same time, good dispersibility and stability in water, and good elasticity.

### SUMMARY OF THE INVENTION

In one aspect the invention is a method for preparing an aqueous dispersion of film-forming cationic polyurethanes comprising: A) reacting one or more aliphatic diisocyanates with an aliphatic tertiary amine having two hydroxyl groups that react with —NCO groups and a linear hydroxyl terminated or polyether or polyester polyol having a molecular weight of from about 500 to about 5,000, to obtain a prepolymer having free isocyanate groups; B) salifying amine groups of the prepolymer with methanesulfonic acid; and C) extending the obtained cationic prepolymer with water. In step A), the molar ratio between aliphatic diisocyanates and compounds having two reactive hydroxyl groups (linear polyol and aliphatic tertiary amine) is from about 1.2 to about 2.2

In another aspect, the invention is a composition comprising an aqueous dispersion of film forming cationic polyurethanes prepared by a method comprising: A) reacting one or more aliphatic diisocyanates with an aliphatic tertiary amine having two hydroxyl groups that react with —NCO groups and a linear polyol of the polyol polyether or polyol polyester type having a molecular weight of from 500 to 5,000, to obtain a prepolymer having free isocyanate groups; B) salifying amine groups of the prepolymer with methanesulfonic acid; and C) extending the obtained cationic prepolymer with water.

In yet another aspect, the invention is a lotion useful as a hair dressing comprising a solution of the composition comprising an aqueous dispersion of film forming cationic polyurethanes prepared by a method comprising: A) reacting one or more aliphatic diisocyanates with an aliphatic tertiary amine two hydroxyl groups that react with an —NCO group and a linear polyol of the polyol polyether or polyol polyester type having a molecular weight of from 500 to 5,000, to obtain a prepolymer having free isocyanate groups; B) salifying amine groups of the prepolymer with methanesulfonic acid; and C) extending the obtained cationic prepolymer with water, wherein the composition is dissolved in a solvent selected from the group consisting of water, ethanol, and combinations thereof, wherein the solids content of the solution ranges from about 0.1 to about 30% by weight of the polyurethane.

In another aspect, the invention is a method of treating leather comprising applying to the surface of a leather substrate a lotion comprising a solution of the composition useful for treating paper, leather and textiles comprising an aqueous dispersion of film forming cationic polyurethanes prepared by a method comprising: A) reacting one or more aliphatic diisocyanates with an aliphatic tertiary amine two hydroxyl groups that react with an —NCO group and a linear polyol of the polyol polyether or polyol polyester type having a molecular weight of from 500 to 5,000, to obtain a prepolymer having free isocyanate groups; B) salifying amine groups of the prepolymer with methanesulfonic acid; and C) extending the obtained cationic prepolymer with water, wherein the aqueous composition contains from about 0.1 to about 30% by weight of the polyurethane and wherein the composition is dissolved in a solvent selected from the group consisting of water, ethanol, and combinations thereof. In step A), the molar ratio between aliphatic diisocyanates and compounds having two reactive hydroxyl groups (linear polyol and aliphatic tertiary amine) is from about 1.2 to about 2.2

In still another aspect, the invention is a method of treating paper comprising applying to paper fibers or the surface of a paper substrate a lotion comprising a solution of an aqueous dispersion of film forming cationic polyurethanes prepared by a method comprising: A) reacting one or more aliphatic diisocyanates with an aliphatic tertiary amine two hydroxyl groups that react with an —NCO group and a linear polyol of the polyol polyether or polyol polyester type having a molecular weight of from 500 to 5,000, to obtain a prepolymer having free isocyanate groups; B) salifying amine groups of the prepolymer with methanesulfonic acid; and C) extending the obtained cationic prepolymer with water, wherein the aqueous composition contains from about 0.1 to about 30% by weight of the polyurethane and wherein the composition is dissolved in a solvent selected from the group consisting of water, ethanol, and combinations thereof. In step A), the molar ratio between aliphatic diisocyanates and compounds having two reactive hydroxyl groups (linear polyol and aliphatic tertiary amine) is from about 1.2 to about 2.2

In another aspect, the invention is a method of treating hair comprising applying to hair a hair dressing comprising a solution of a composition comprising an aqueous dispersion of film forming cationic polyurethanes prepared by a method comprising: A) reacting one or more aliphatic diisocyanates with an aliphatic tertiary amine two hydroxyl groups that react with an —NCO group and a linear polyol of the polyol polyether or polyol polyester type having a molecular weight of from 500 to 5,000, to obtain a prepolymer having free isocyanate groups; B) salifying amine groups of the prepolymer with methanesulfonic acid; and C) extending the obtained cationic prepolymer with water, wherein the aqueous composition contains from about 0.1 to about 30% by weight of the polyurethane and wherein the composition is dissolved in a solvent selected from the group consisting of water, ethanol, and combinations thereof. In step A), the molar ratio between aliphatic diisocyanates and compounds having two reactive hydroxyl groups (linear polyol and aliphatic tertiary amine) is from about 1.2 to about 2.2

### DESCRIPTION

For the purposes of this application, the term molecular weight, when referred to polyols, means molecular weight as calculated using the hydroxyl number of a polyol. This molecular weight may be determined:

$$molecular weight = \frac{17 * 3300 * functionality of the polyol}{OH}$$

wherein the analysis of OH is done by titration of the hydroxyl number (noted OH) in mg/g as potassium hydroxide, according to the ASTM method D4274

The Applicant has now found that remarkably stable aqueous dispersions of film-forming cationic polyurethanes can be obtained by using in their preparation aliphatic diisocyanates and methanesulfonic acid as the salifying agent of the tertiary amine groups of the polymer.

The improved stability of the aqueous dispersions, beside providing positive effects on their storability, also advantageously increases the transparency and gloss of the films obtained there from.

According to the Applicant's knowledge, the stabilizing effect of the combination of aliphatic diisocyanate with methanesulfonic acid is not known from the literature, even if methanesulfonic acid is cited in U.S. Pat. No. 3,480,592 (col. 4, I. 35-col. 5, I. 60) among a long list of utilizable compounds as a possible salifying agent for cationic polyurethanes.

Accordingly, the fundamental object of the present invention is an aqueous dispersion of film-forming cationic polyurethanes obtained by: A) reacting one or more aliphatic diisocyanates with an aliphatic tertiary amine having two hydroxyl groups that react with —NCO groups and a linear polyol of the polyol polyether or polyol polyester type having molecular weight from 500 to 5,000, to obtain a prepolymer having free isocyanate groups; B) salifying with methanesulfonic acid the amine groups of the prepolymer; C) extending the thus obtained cationic prepolymer with water.

In step A), the molar ratio between aliphatic diisocyanates and compounds having two reactive hydroxyl groups (linear polyol and aliphatic tertiary amine) is from 1.2 and 2.2, and preferably from 1.3 to 1.8.

The cationic polyurethane of the present invention has preferably a weight average molecular weight from 2,000 to 50,000, as determined by Gel Permeation Chromatography.

The diisocyanates that can be used according to the present invention are aliphatic; with the term “aliphatic” we mean both aliphatic and cycloaliphatic; preferably the diisocyanates are chosen among 4,4′-dicyclohexyl methane-diisocyanate, 1-isocyanate, 3-isocyanate-methyl-3,5,5-trimethylcyclohexane (or isophorone diisocyanate), hexamethylene diisocyanate and mixtures thereof.

The aliphatic amine having two hydroxyl groups that react with the —NCO group is preferably 3-(diethylamino)-1,2-propanediol or an alkyl diethanol amine; among the alkyl diethanol amines, methyl-, ethyl, isopropyl-, n-butyl-, t-butyl-, cyclohexyl-, n-hexyl-diethanolamine, and mixtures thereof are particularly useful.

The amount of amine which is bound in the polyurethane is measured in milliequivalent of amine and is calculated as the percentage of the ratio of amine millimoles on grams of dry polyurethane.

In the present invention the milliequivalents of amine in the polyurethanes range from 20 to 150, preferably from 30 to 90.

The linear polyols that may be used for the invention have molecular weight Mₙ from 500 to 5,000, preferably from 800 to 3,000 and they are hydroxyl terminated polyether or polyester diols.

Examples of polyol polyester that may be used according to the present invention are the products obtained from the polycondensation of acids or anhydrides, having from 4 to 9 carbon atoms and preferably of the aliphatic kind, with aliphatic diols having from 2 to 8 carbon atoms.

Among the preferred polyol polyester we cite polyadipate from 1,4-butandiol and ethylene glycol, polyadipate from 1,6-hexanediol and neopentylglycol, polyadipate phthalate from 1,6 hexanediol, polycaprolactones and polycarbonate diols from 1,6-hexanediol and 1,4-butanediol.

Polyadipate from 1,4-butandiol and ethylene glycol, polyadipate from 1,6-hexanediol and neopentylglycol, polyadipate phthalate from 1,6 hexanediol are particularly preferred.

Examples of polyol polyethers that may be used are polyethylene glycol, polytetrahydrofuran, and mixtures thereof; the latter is preferred for the realization of the present invention.

Mixtures of different linear polyols may be used in step A).

In the most common embodiments, step A) is carried out without any solvent, but, preferably, at the end of step A) and before conducting the salification of step B) a non reactive organic solvent, by way of example N-methylpyrrolidone or acetone, is added.

The salification is preferably carried out at room temperature by using methanesulfonic acid in one of its more common commercially available forms, that is as a 70% w/w aqueous solution.

The salification step is made by adding methanesulfonic acid in molar amounts ranging from 60 to 120%, more preferably from 95 to 105%, the percentage being referred to the moles of tertiary amine.

The extension of the cationic polyurethane obtained after step B) is preferably made by reaction with water at temperature of 20-60° C.

Typically, in the final aqueous dispersion the content of film-forming cationic polyurethane is from 20 to 35% by weight.

This range of concentrations is particularly appreciable for industrial use because it corresponds to sufficiently concentrated dispersions that avoid the useless transportation of large amounts of water and; at the same time, the viscosity of the dispersions in this range of concentrations allows their easy handling.

Moreover, according to here described invention, it is also possible to obtain cationic dispersions that are free from solvents and have low content of volatile organic compounds.

The aqueous dispersions of film-forming polyurethanes of the present invention are advantageously used in the preparation of cosmetic products, such as make up products (mascaras, long lasting lipsticks), hair lacquers, nail varnishes, hair gels, waterproof sunscreen creams.

The cationic nature of the dispersions of the present application makes them particularly affine to keratin and therefore to hair; they are advantageously employed in products for the treatment and care of hair. As illustrated in the Examples, the dispersions of the application are useful for imparting softness and improved styling.

Beside the cosmetic field, the dispersions of the invention are useful in leather finishing, in textile sizing and finishing, in the coating of paper, wood and plastic, as felting agents for wool. Particularly when used in leather finishing, the dispersions of the invention are superior to conventional products, but they are also especially useful as primers.

When used in the production of paper and textiles, the dispersions of the invention are especially useful when used in formulations that are not compatible with anionic binders, for examples in acidic formulations and in formulations comprising metal cations.

When the aqueous dispersion of film-forming polyurethanes described are used as hair dressings with film-forming properties, they are usually employed in the form of aqueous or ethanolic solutions (lotions). These lotions contain from 0.1 to 30, preferably from 1 to 15% by weight of the polyurethane.

When the aqueous dispersion of film-forming polyurethanes described are used for treating leather, paper or textiles they are usually employed in the form of aqueous composition (solutions or dispersions). These solutions or dispersions contain from 0.1 to 30, preferably from 1 to 15% by weight of the polyurethane and are particularly useful for the following treatments: finishing or coating leather; sizing or coating paper; sizing or finishing or printing textiles.

In the following examples the preparation of aqueous dispersions according to the present invention is reported (Ex. I-III, V) and of comparative dispersions (Ex. IV, VI-VIII); the reported percentages are by weight.

In an applicative example the preparation of a hair lacquer from an aqueous dispersion according to the invention is described.

The present invention thus also provides cosmetic preparations comprising an effective amount of the aqueous dispersion of film-forming polyurethanes described above. An effective amount generally ranges, depending on the use, from 1 to 50% by weight, in particular from 5 to 30% by weight, based on the preparation.

### EXAMPLES

In the examples the following materials were used:
Polyol 1=polyadipate from 1,4-butanediol and ethylene glycol having molecular weight 830 g/mol;
Polyol 2=polytetramethylene ether glycol having molecular weight 1000 g/mol;
Amine 1=methyldietanol amine (MW 119.16 g/mol);
Amine 2=3-(diethylamino)-1,2-propanediol (MW 147.22 g/mol);
Acid 1=methanesulfonic acid (MW 96.11 g/mol), 70% aqueous solution;
Acid 2=p-toluenesulfonic acid (MW 172.2 g/mol), 20% aqueous solution;
Acid 3=phosphoric acid (MW 98.00 g/mol), 75% aqueous solution;
Acid 4=formic acid (MW 46.03 g/mol), 85% aqueous solution;
Diisocyanate 1=4-4′-methylene-bis-(4-cyclohexyl isocyanate), MW 262.35 g/mol (Desmodur W, Bayer);
Diisocyanate 2=Hexamethylene diisocyanate, MW 168.19 (Desmodur I, Bayer); and
Diisocyanate 3=Toluene diisocyanate, MW 174.16 (Desmodur T80, Bayer).

### Example I

Preparation of a cationic polyurethane dispersion according to the invention.

A reaction vessel, equipped with internal thermometer, stirrer and cooler, was filled, under nitrogen atmosphere and at room temperature, with 160.21 g (160 mmol) of Polyol 2 (fed at 40° C.), 17.87 g (150 mmol) of Amine 1 and 0.048 g of benzoyl chloride. The mixture was heated to 40° C. and stirred for 30 minutes. 121.91 g (465 mmol) of Diisocyanate 1 were added under stirring to the homogeneous mixture which was then maintained at 60° C. for 30 minutes. The reaction temperature was brought to 90° C. until the titrimetric determination of the free —NCO groups still present gave a calculated value of 4.35% by weight (value determined in this example as well as in other examples according to the standard method ASTM D2572).

Once obtained the above NCO value, the prepolymer was cooled to 60° C. adding at the same time 105 g of acetone. At about 40° C., 20.59 g (150 mmol) of Acid 1 were added under stirring and after 10 minutes, at temperature of 40° C., 679.3 g of demineralized water were added.

Afterwards acetone was distilled off under vacuum while keeping the cationic waterborne polyurethane dispersion under stirring to obtain a stable translucent product with 30% solid content.

At the end of the distillation process the —NCO peak in the IR spectrum at 2240 cm-1 had disappeared.

The obtained cationic aqueous polyurethane dispersion is stable for more than 6 months.

### Examples II-VIII

The procedure of Example I was followed while changing the ratios and raw materials as reported in Table 1a (examples according to the invention) and in Table 2a (comparative examples).

Tables 1b and 2b report the peculiarities of the polyurethane dispersions characteristics.

TABLE 1a
(amount of raw materials)

| | Example I | | Example II | | Example III | | Example V | |
|---|---|---|---|---|---|---|---|---|
| | g | mmol | g | mmol | g | mmol | g | mmol |
| POLYOL 1 | — | | 168.34 | 203 | 173.57 | 209 | — | — |
| POLYOL 2 | 160.2 | 160 | — | — | — | — | 177 | 177 |
| AMINE 1 | 17.87 | 150 | 17.87 | 150 | — | — | 19.66 | 165 |
| AMINE 2 | — | | — | — | 22.08 | 150 | — | — |
| DIISOCYANATE 1 | 121.91 | 465 | 44.45 | 169 | 40.77 | 155 | 40.25 | 153 |
| DIISOCYANATE 2 | — | | 69.33 | 412 | 63.58 | 378 | 62.77 | 373 |
| ACID 1 | 20.59 | 150 | 20.59 | 150 | 20.59 | 150 | 22.65 | 165 |
| DEMINERALIZED WATER | 679.25 | | 679.25 | | 679.0 | | 670 | |
| ACETONE | 105 | | 110 | | 110 | | 110 | |

TABLE 1b
Dispersions characteristics

| | Example I | Example II | Example III | Example V |
|---|---|---|---|---|
| % NCO | 4.35% | 4.94% | 4.53% | 3.84% |
| PREPOLYMER | | | | |
| SOLID | 30% | 30% | 30% | 30% |
| CONTENT | | | | |
| SHELF LIFE | >6 months | >6 months | >6 months | >6 months |

TABLE 2a
(amounts of raw materials)

| | Example IV* | | Example VI* | | Example VII* | | Example VIII* | |
|---|---|---|---|---|---|---|---|---|
| | g | mmol | g | mmol | g | mmol | g | mmol |
| POLYOL 1 | — | — | — | — | — | — | — | — |
| POLYOL 2 | 160.21 | 160 | 227.5 | 227 | 175.51 | 175 | 161.81 | 162 |
| AMINE 1 | 17.87 | 150 | 16.68 | 140 | 18.3 | 153 | 15.01 | 126 |
| DIISOCYANATE 1 | 121.91 | 465 | — | — | 41.46 | 158 | — | — |
| DIISOCYANATE 2 | — | — | 83.35 | 495 | 64.72 | 384 | — | — |
| DIISOCYANATE 3 | — | — | — | — | — | — | 75.11 | 431 |
| ACID 1 | — | — | — | — | — | — | 17.29 | 126 |
| ACID 2 | 129.0 | 150 | — | — | — | — | — | — |
| ACID 3 | — | — | — | — | 20.00 | 153 | — | — |
| ACID 4 | — | — | 7.49 | 138 | — | — | — | — |
| DEMINERALIZED WATER | 545.0 | | 677.0 | | 680.0 | | 573 | |
| ACETONE | 105 | | 105 | | 110 | | 125 | |

*comparative

TABLE 2b
Characteristics of the Dispersions

| | | | Example | Example |
|---|---|---|---|---|
| | Example IV* | Example VI* | VII* | VIII* |
| % NCO | 4.35% | 3.3% | 4.5% | 4.8% |
| PREPOLYMER | | | | |
| SOLID | N.D. | 33% | 30% | N.D. |
| CONTENT | | | | |
| SHELF LIFE | 30′ | 60′ | 80′ | 0** |

*comparative
**the dispersion separates during its preparation process.

### Application Test

Two standard formulations were prepared to evaluate the performances of the product obtained from Example I in comparison with a standard film forming polymer used in cosmetic for hair styling. The comparative polymer used was a polyvinylpyrrolidone/vinylacetate (CTFA name: VP/VA Copolymer, LUVISKOL® VA64 P from BASF Aktiengesellschaft)

The formulations have the following composition (a.c.=active content).

### Formulation 1:

| | |
|---|---|
| Dispersion obtained by Example 1 (a.c. 30% w/w) | 6% |
| Water/Ethanol Mixture 1/2 | to 100% |

### Formulation 2:

| | |
|---|---|
| LUVISKOL VA64 P (a.c. >95% w/w) | 1.8% |
| Water/Ethanol Mixture 1/2 | to 100% |

The two film forming polymers, the polyurethane from Example I and Luviskol VA64 P, are completely dissolved into the Water/Ethanol mixture and Formulation 1 and 2 are transferred into spray containers.

To test their film forming effectiveness, 1 g of each Formulation is sprayed on a lock and dried for 1 hr. A panel test carried out on 10 persons showed that the finishing obtained with Formulation 1 is softer.

To test the curl retention, 1 g of each Formulation is sprayed onto a lock of medium/long length and set with a curler laying for 1 hr. A panel test carried out on 10 persons showed that the finishing obtained with Formulation 1 is more natural and the lock is better styled.

It is therefore pointed out that the polyurethane dispersion obtained from Example I shows good finishing performance and set on hair.

What is claimed is:

1. A method for preparing an aqueous dispersion of film-forming cationic polyurethanes comprising:
A) reacting one or more aliphatic diisocyanates with an aliphatic tertiary amine having two hydroxyl groups that react with an —NCO group and a linear hydroxyl terminated polyether or polyester dioll having a molecular weight of from about 500 to about 5,000, to obtain a prepolymer having free isocyanate groups;
B) salifying amine groups of the prepolymer with methanesulfonic acid; and
C) extending the obtained cationic prepolymer with water, and,
wherein in step A), the molar ratio between aliphatic diisocyanates and compounds having two reactive hydroxyl groups (linear polyol and aliphatic tertiary amine) is from about 1.2 to about 2.2.

2. The method of claim 1, wherein the concentration of film-forming cationic polyurethanes is from about 20% to about 35% by weight.

3. The method of claim 1, wherein the salifying comprises adding methanesulfonic acid in molar amounts ranging from about 60% to about 120% moles per mole of a tertiary amine.

4. The method of claim 3, wherein the extending of the cationic prepolymer comprises a reaction with water at a temperature of from about 20° to about 60° C.

5. The method of claim 1, wherein the molar ratio between the aliphatic diisocyanates and the sum of the moles of linear polyol and the aliphatic tertiary amine is from about 1.2 to about 2.2.

6. The method of claim 5, wherein the aliphatic diisocyanates are selected from the group consisting of 4,4′dicyclohexyl methane-diisocyanate, 1-isocyanate, 3-isocyanatemethyl-3,5,5-trimethylcyclohexane (or isophorone diisocyanate), hexamethylene diisocyanate and combinations thereof.

7. The method of claim 6, wherein the aliphatic tertiary amine having two hydroxyl groups that react with the —NCO group is 3-(diethylamino)-1,2-propanediol or an alkyl diethanol amine.

8. The method of claim 7, wherein the alkyl diethanol amine is selected from the group consisting of methyl diethanolamine, ethyl diethanolamine, isopropyl diethanolamine, n-butyl diethanolamine, t-butyl diethanolamine, cyclohexyl diethanolamine, n-hexyldiethanol amine, and combinations thereof.

9. The method of claim 1, wherein the polyol is a polyadipate prepared using 1,4-butandiol and ethylene glycol, a polyadipate prepared using 1,6-hexanediol and neopentylglycol, or a polyadipate phthalate prepared using 1,6 hexanediol.

10. The method of claim 1, wherein the polyol is polytetrahydrofuran.

11. A composition comprising an aqueous dispersion of film forming cationic polyurethanes prepared by a method comprising:
A) reacting one or more aliphatic diisocyanates with an aliphatic tertiary amine having two hydroxyl groups that react with an —NCO group and a linear hydroxyl terminated diol polyol of the polyether or polyol polyester type having a molecular weight of from 500 to 5,000, to obtain a prepolymer having free isocyanate groups;
B) salifying amine groups of the prepolymer with methanesulfonic acid; and
C) extending the obtained cationic prepolymer with water, and,
wherein in step A), the molar ratio between aliphatic diisocyanates and compounds having two reactive hydroxyl groups (linear polyol and aliphatic tertiary amine) is from about 1.2 to about 2.2.

12. The composition of claim 11, wherein the content of film-forming cationic polyurethanes is from about 20% to about 35% by weight.

13. The composition of claim 11, wherein the salifying comprises adding methanesulfonic acid in molar amounts ranging from about 60% to about 120% moles per mole of a tertiary amine.

14. The composition of claim 13, wherein the extending of the cationic prepolymer comprises a reaction with water at a temperature of from about 20° to about 60° C.

15. The composition of claim 11, wherein the molar ratio between the aliphatic diisocyanates and the sum of the moles of linear polyol and the aliphatic tertiary amine is from about 1.2 to about 2.2.

16. The composition of claim 15, wherein the diisocyanates are selected from the group consisting of 4,4′dicyclohexyl methane-diisocyanate, 1-isocyanate, 3-isocyanatemethyl-3,5,5-trimethylcyclohexane (or isophorone diisocyanate), hexamethylene diisocyanate and combinations thereof.

17. The composition of claim 16, wherein the aliphatic tertiary amine having two hydroxyl groups that react with the —NCO group is 3-(diethylamino)-1,2-propanediol or an alkyl diethanol amine.

18. The composition of claim 17, wherein the alkyl diethanol amine is selected from the group consisting of methyldiethanol amine, ethyl diethanolamine, isopropyl diethanolamine, n-butyl diethanolamine, t-butyl diethanolamine, cyclohexyldiethanolamine, n-hexyldiethanol amine, and combinations thereof.

19. The composition of claim 11, wherein the polyol is a polyadipate from 1,4-butandiol and ethylene glycol, polyadipate from 1,6-hexanediol and neopentylglycol, or polyadipate phthalate from 1,6 hexanediol.

20. The composition of claim 11, wherein the polyol is polytetrahydrofuran.

21. A method of treating leather comprising applying to the surface of a leather substrate a dispersion comprising a solution of the composition of claim 11 wherein the composition of claim 11 is dissolved in a solvent selected from the group consisting of water, ethanol, and combinations thereof, wherein the solids content of the solution ranges from about 0.1 to about 30% by weight of the polyurethane.

22. A method of treating paper comprising applying to paper fibers or the surface of a paper substrate a dispersion comprising a solution of the composition of claim 11 wherein the composition of claim 11 is dissolved in a solvent selected from the group consisting of water, ethanol, and combinations thereof, wherein the solids content of the solution ranges from about 0.1 to about 30% by weight of the polyurethane.

23. A method of treating textile fibers or textiles comprising applying to textile fibers or the surface of a textile substrate a dispersion comprising a solution of the composition of claim 11 wherein the composition of claim 11 is dissolved in a solvent selected from the group consisting of water, ethanol, and combinations thereof, wherein the solids content of the solution ranges from about 0.1 to about 30% by weight of the polyurethane.

24. A method of treating hair comprising applying to hair a hair dressing comprising a solution of the composition of claim 11 wherein the composition of claim 11 is dissolved in a solvent selected from the group consisting of water, ethanol, and combinations thereof, wherein the solids content of the solution ranges from about 0.1 to about 30% by weight of the polyurethane.
